(19) United States
(12) Patent Application Publication
(10) Pub. No.: US 2006/0234337 A1
(43) Pub. Date: Oct. 19, 2006

(54) Expression system for actinomycete-origin cytochrome p-450 in escherichia coli

(76) Inventors: Akira Arisawa, Iwata-shi (JP); Ayako Kumeda, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 10/510,826

(22) PCT Filed: Apr. 11, 2003
(86) PCT No.: PCT/JP03/04609

§ 371 Date: Oct. 12, 2004

(30) Foreign Application Priority Data
Apr. 12, 2002 (JP) 2002-110311

(51) Int. Cl. C12P 21/06; C12N 9/02; C07H 21/04; C12N 15/74; C12N 1/21

(52) U.S. Cl. 435/69.1; 435/189; 435/488; 435/252.33; 536/23.2

(57) ABSTRACT

This invention relates to a system for the expression of cytochrome P-450 gene in host Escherichia coli, and provides Escherichia coli which contains actinomycete ferredoxin gene and also ferredoxin gene and ferredoxin reductase gene which are xenogenic to Escherichia coli. Thus, this invention is useful for the promotion of effective single oxygen atom insertional reaction of a substrate organic compound.

### TECHNICAL FIELD

This invention relates to a system for the expression of actinomycete cytochrome P-450 genes in Escherichia coli.

### BACKGROUND TECHNOLOGY

Cytochrome P-450 enzymes (hereinafter referred to simply as “P-450s”) which are encoded by cytochrome P-450 genes are a general name of a group of protoheme-containing proteins whose reduced form shows Soret band around 450 nm when bound to carbon monoxide. P-450s are bound to microsome in tissue of various kinds of animal or plant, or in fungi or yeasts, or to inner membrane of mitochondrion in tissue of some kind of animals. In certain kinds of bacteria and fungi, P-450s exist in soluble state.

P-450s show various types of substrate-specificity. Some P-450s have abnormally so wide substrate-specificity that they can react with various kinds of organic compounds as substrate. Some, on the other hand, have considerably strict substrate-specificity, and react only with comparatively limited kinds of organic compounds. P-450s also show excellent stereo- and/or regio-specificity to reaction site. With regard to concrete functions, P-450s are known to catalyze reactions such as hydroxylation, epoxidation, dealkylation and denitrification, of xenobiotics in a cell which expresses said P-450s. For example, most of drugs which are administered to human are metabolized and inactivated in the body by various or specific action, such as hydroxylation, of P-450. In some cases, on the contrary, pharmacological effects of the drugs are improved, or subsidiary action is enhanced. P-450 is, therefore, medically very important from the viewpoint of the research of metabolism of medicine or the development of prodrugs.

Thus, P-450s which have drug-metabolizing functions for higher organisms including human have been studied from every angle for long years. Although these enzymes are obtained from microsome fractions of liver of higher organisms, it is difficult to purify these enzymes into single isozyme. On this account, there has been developed a technology to express functionally a gene which encodes single isozyme in a host such as Escherichia coli or yeast, and to thus conveniently investigate the metabolic role of the enzyme.

P-450s of higher organisms which have such drug-metabolizing functions as mentioned above have never been successfully applied to material production on industrial scale. P-450s of higher organisms, when functionally expressed in a host such as Escherichia coli or yeast, show only low productivity as compared with P-450s of bacteria, and also cause various side reactions. For these reasons, P-450s of higher organisms have been used only restrictively.

In the case of P-450 originated from microorganisms such as fungi and bacteria, on the other hand, some kinds of P-450s are known to serve for the production of industrially useful materials. Some of such kinds of P-450s have actually been utilized for industrial production of useful medicine. Typical example is the production of pravastatin, a medicine to remedy hyperlipidemia, by means of the hydroxylation at 6β-position of compactin with Storeptomyces carhophigus, a species of actinomycete (Watanabe et al., Gene, 163 (1995) 81-85, Japanese Patent Application Laid-Open (Kokai) Publication No. Hei 6 (1994)-70780). There has also been put into practice a method by which to produce active vitamin D₃ by means of the hydroxylation at 1α- and 25-positions of vitamin D₃ with use of Pseudonocardia autotrophica, a species of actinomycete.

Such microbial conversion of drugs with use of actinomycete cytochrome P-450 enzymes as mentioned above have been carried out with use of culture liquids or cells of actinomycetes which express said enzymes. There have also been used culture liquids wherein genes which encode actinomycete P-450s have been introduced into Storeptomyces lividans, that is also a species of actinomycete and that is suitable as a host, to express the enzymatic activity. The microbial conversion of substrate compounds with the actinomycete strains which have such genes as mentioned above takes considerable time for the cultivation of the strains and for the conversion of substrate compounds into desired products. Furthermore, some enzymes need consideration on expression-inducible conditions under which to increase the amount of enzyme expressed. Moreover, some species of actinomycetes which are to be used for the conversion have, in themselves, a system for metabolizing or degrading substrate or desired product, which causes the formation of by-products or the reduction of substrate or desired product, and thus decreases the productivity of desired product.

There is also a report of experiment wherein, after the example of functionally expressing a gene which encodes single isozyme of the above-mentioned higher organism-originated P-450s in a host of microorganism such as Escherichia coli, CYP105D1 gene which is a Storeptomyces griseus originated cytochrome P-450 gene was functionally expressed in Escherichia coli (Taylor et al., Biochemical and Biophysical Research Communications (1999) 263: 838-842). It seems that, in this expression system, some suitable electron donor for the P-450 in periplasm of Escherichia coli hydroxylates hydrocarbons in cooperation with the P-450 (Kaderbhai et al, Applied and Environmental Microbiology, 67 (2001) 2136-2138). Such a P-450 gene-expressing system has a merit that Escherichia coli as a host needs less time for cultivation as compared with actinomycetes or the like.

### DISCLOSURE OF INVENTION

The above-mentioned system for the conversion of organic compounds with microorganisms which have P-450s is intended to be used, for instance, for the application to biocatalyst or for the research of drug metabolism. In consideration of application to biocatalyst, in particular, more efficient bioconversion would be demanded. In order to achieve the efficient screening of industrially important and desired actinomycete P-450 enzymes, there would be needed a suitable gene library as an object of robotized enzyme-assaying operation or other convenient and rapid enzyme-assaying operation in high throughput screening or the like. Concretely, there is demanded a library which has actinomycete-originated different cytochrome P-450 genes (actinomycete cytochrome P-450-expression library) and wherein microorganism, preferably handy and quick-growing microorganism, is used as a host in which each constituent clone is capable of expression.

In order to attain the above-mentioned objective, it would be useful to use, as a host, Escherichia coli which, at least, needs comparatively short time for cultivation, and which is considered to have only a few systems for the metabolism or degradation of substrate compounds or products from said substrate compounds. It has been confirmed, however, that only introducing an actinomycete P-450 gene into a host Escherichia coli and cultivating the same, as in the above-mentioned Taylor et al wherein Escherichia coli is used as a host, does not achieve the functional expression of most of various kinds of P-450 genes which are originated from other species of actinomycete (or, in other words, enzymatic activity of P-450 is not shown as expected). Thus, the inventors of the present invention have studied how to construct a system which is capable of functionally expressing actinomycete-originated various kinds of P-450 genes surely and with high enzymatic activity. As a result, they have found out that, when a certain electron transport system originated from microorganism which is xenogenic to Escherichia coli introduced together with P-450 gene and is then allowed to co-express, the P-450 gene originated from various species of actinomycete is functionally expressed.

Based on such a finding as mentioned above, this invention provides a system for the expression of actinomycete cytochrome P-450 genes in host Escherichia coli, wherein said Escherichia coli supports a recombinant DNA molecule which comprises xenogenic microorganism-originated ferredoxin gene, ferredoxin reductase gene and said cytochrome P-450 gene in operable state.

Such an expression system as explained above is capable of the expression of such a gene as mentioned in the above Taylor et al. which encodes actinomycete cytochrome P-450, and which is incapable of conjugating native electron transport system of Escherichia coli. In other words, the expression system of this invention achieves expected enzymatic activity of P-450 whether P-450 gene may conjugate native electron transport system of Escherichia coli or not. Thus, the term “functionally express” in this specification means that a gene of interest expresses protein, which is encoded by the gene, in an active form.

In the following, this invention is explained in more detail.

Host Escherichia coli means certain kinds of Escherichia coli which are usable for the propagation of vectors such as plasmids and phages and of inserted genes. Any species of host will do if only, in a recombinant DNA experiment with use of host-vector system, a vector with an exogenous DNA fragment is capable of replication after transformation. As a host for said host-vector system, Escherichia coli on the market would be conveniently utilized.

Actinomycete cytochrome P-450 genes in this invention include P-450 genes originated from any genus of bacteria that belong to order Actinomycetales if only the bacteria have P-450 genes which serve to achieve the objective of this invention in some form or other (e.g. on chromosome or plasmid). Thus, cytochrome P-450 genes include all that encodes protein mentioned above which has such activity as to catalyze single oxygen atom insertional reaction in accordance with this invention. Although not restrictive, examples of P-450 genes which are intended to be incorporated into the expression system of this invention include those which have the above-mentioned function, at least a part of whose DNA sequences have been determined, and each of whose sequence information is available from gene data base (EMBL and GenBank), concretely, those which are originated from the following species of actinomycte and which encode protein having the above-mentioned activity, or those which have functions of cytochrome P-450 as mentioned below.

| Actinomycete | Function of cytochrome P-450 |
|---|---|
| Amycolatopsis orientalis | Unknown |
| Actinomadura verrucosospora | Biosynthesis of vercopeptin |
| Amycolata autotrophica | Unknown |
| Amycolatopsis mediterranei | Biosynthesis of rifamycin |
| Amycolatopsis mediterranei | Biosynthesis of balhimycin |
| Kitasatospora griseospola | Biosynthesis of terpentecin |
| Micromonospora griseorubida | Biosynthesis of mycinamicin |
| Micromonospora inyoensis | Unknown |
| Microtetraspora recticatena | Hydroxylation of compactin |
| Mycobacterium smegmatis mc2155 | Degradation of piperidine and |
| | pyrrolidine |
| Mycobacterium sp. FM10 | Unknown |
| Mycobacterium tuberculosis H37Rv | 22 P-450 genes in whole genome |
| | (function unknown) |
| Myxococcus xanthus | Polyketide antibiotic TA |
| Pseudonocardia autotrophica | Hydroxylation of vitamin D₃ |
| (old name: Amycolata autotrophica) | |
| Rhodococcus erythropolis | Degradation of thiocarbamate |
| | herbicide |
| Rhodococcus fascians (D188) | Synthesis of phytophysiologically |
| | active substance |
| Rhodococcus ruber | Degradation of ethyl-tert-butyl |
| | ether |
| Saccharopolyspora erythraea | Hydroxylation of erythromycin |
| Streptoalloteichus hindustanus | Unknown |
| Streptomyces acidiscabies | Biosynthesis of thaxtomin A |
| Streptomyces albus | Unknown |
| Streptomyces ansochromogenes | Biosynthesis of nikkomycin |
| Streptomyces antibioticus | Biosynthesis of oleandomycin |
| Streptomyces antibioticus | Biosynthesis of simocyclinone |
| Streptomyces aureofaciens Ren71 | Unknown |
| Streptomyces avermitilis | Formation of furan ring of |
| | avermectin |
| Streptomyces avermitilis | Biosynthesis of oligomycin |
| Streptomyces avermitilis | Biosynthesis of polyketide-4 |
| Streptomyces avermitilis | Biosynthesis of polyketide-9 |
| Streptomyces avermitilis | Biosynthesis of other type |
| | polyketide |
| Streptomyces avermitilis | Biosynthesis of polyene |
| | macrolide |
| Streptomyces avermitilis | Biosynthesis of peptide-7 |
| Streptomyces carbophilus | Hydroxylation of compactin |
| Streptomyces clavuligerus | Biosynthesis of clavulanic acid |
| Streptomyces coelicolor A3(2) | 18 P-450 genes in whole genome |
| | (function unknown) |
| Streptomyces fluvus | Hydroxylation of compactin |
| Streptomyces fradiae | Biosynthesis of tylosin |
| Streptomyces glaucescens | Unknown |
| Streptomyces griseolus | Degradation of sulfonylurea |
| | herbicide |
| Streptomyces griseus | Unknown |
| Streptomyces hygroscopicus | Biosynthesis of rapamycin |
| Streptomyces hygroscopicus | Biosynthesis of FK520 |
| var. ascomyceticus | |
| Streptomyces lavendulae | Biosynthesis of mitomycin |
| Streptomyces lavendulae | Biosynthesis of complestatin |
| Streptomyces lividans | Unknown |
| Streptomyces maritimus | Biosynthesis of enterocin |
| Streptomyces natalensis | Biosynthesis of pimaricin |
| Streptomyces nodosus | Biosynthesis of amphotericin |
| Streptomyces nogalater | Biosynthesis of nogalamycin |
| Streptomyces noursei | Biosynthesis of nystatin |
| Streptomyces peucetius | Hydroxylation of daunomycin |
| Streptomyces peucetius | Hydroxylation of daunomycin |
| subsp. caesius | |
| Streptomyces rishiriensis | Biosynthesis of coumermycin A1 |
| strain DSM 40489 | |
| Streptomyces sclerotialus | Unknown |
| Streptomyces sp. | Hydroxylation of FK-506 |
| Streptomyces sp. | Unknown |
| Streptomyces spheroids | Biosynthesis of novobiocin |
| Streptomyces tendae | Biosynthesis of nikkomycin |
| Streptomyces thermotolerans | Epoxidation of carbomycin |
| Streptomyces venezuelae | Biosynthesis of pikromycin, |
| | methymycin |

The following actinomycete P-450 are also included as usable in this invention. Each of the following literatures gives guidance how to prepare gene which encodes each enzyme.

Compactin-hydroxylating enzyme originated from Streptomyces carbophilus (P-450_{sca}-2):

Watanabe et al., Gene 163 (1995) 81-85 or Japanese Laid-Open (Kokai) Patent Publication No. Hei 6 (1994)-70780
   Microtetraspora recticatena:
Japanese Laid-Open (Kokai) Patent Publication No. 2001-286293
   Vitamin D3-hydroxylating enzyme originated from Amycolata sp.:
Sasaki et al., Applied Microbiology and Biotechnology (1992) 38: 152-157

Streptomyces roseochromogenes-originated progesterone-hydroxylating enzyme (Berrie et al., Journal of Steroid Biochemistry & Molecular Biology 77 (2001) 87-96) can also be mentioned. Although gene sequence of this Streptomyces roseochromogenes is not mentioned in published literatures, function and biochemical properties of this P-450 enzyme have detailedly determined, and, on the basis of which information, it is easy to prepare gene which encodes said P-450 enzyme.

Cytochrome P-450-encoding genes (or P-450 genes) as mentioned in this invention include any gene so long as it can be isolated from total DNAs of the above-mentioned actinomycetes or can be amplified by PCR reaction, which is mentioned later, on the basis of information of nucleotide sequences of said total DNAs, and so long as it is capable of functional expression in the system of this invention for the expression of P-450 genes. Also included in P-450 genes of this invention are polynucleotides which are functionally equivalent to the above-mentioned genes (also called native gene), and which have activity to catalyze single oxygen atom insertional reaction against corresponding substrates in the expression system of this invention. It is guessed that complement base sequences of said equivalent polynucleotides hybridize with corresponding native genes under a certain hybridization condition, e.g., under stringent condition in 2×SSC (standard saline citrate) at 60° C., preferably in 0.5×SSC at 60° C., most desirably 0.2×SSC at 60° C. When each of the polynucleotides is lined up side by side with the corresponding native gene, there would be shown homology of 80%, preferably 90%, most desirably at least about 95%. This “% homology” means percentage of nucleotide which is in common between two sequences when the two sequences are lined up side by side with each other in an optimum manner. [Thus, “% homology”=(number of coincident positions/total number of positions)×100. This can be calculated with use of algorithm on the market. Such an algorithm is incorporated in NBLAST and XBLAST programs which are mentioned in Altschul et a., J. Mol. Biol. 215 (1990) 403-410.]

Ferredoxin gene which is incorporated in the expression system of this invention is a DNA molecule which is originated from microorganisms (or bacteria) which are xenogenic to host Escherichia coli. Ferredoxin gene generally encodes protein which functions as an electron transporter having a molecular weight of about 6,000 to 14,000. Ferredoxin gene may be originated from any bacteria except Escherichia coli so long as the bacteria participate in the functional expression of P-450 gene when co-expressed with the above-mentioned actinomycete P-450 gene and further with ferredoxin reductase gene which will be mentioned later. Concrete examples of said bacteria, although not restrictive, include actinomycete which may or may not be the same as mentioned above from which P-450 gene is originated.

Also usable is ferredoxin gene originated from bacteria, e.g., of genus Pseudomonas, which belong to different genus from that of actinomycete from which P-450 gene is originated. Examples of such a ferredoxin gene include putidaredoxin gene (also called camB) as mentioned in Peterson et al., The Journal of Biological Chemistry, 265 (1990) 6066-6073.

When ferredoxin gene is originated from the same actinomycete from which P-450 gene is originated, P-450 gene and ferredoxin gene may sometimes constitute a gene cluster in which said P-450 gene and ferredoxin gene exist adjacent to each other on genomic DNA. In such a case, a DNA fragment which contains both of said genes may be used in the expression system of this invention. In the expression system of this invention, ferredoxin gene may exist with another ferredoxin. A preferable example of such a case is the use of ferredoxin gene originated from actinomycete in combination with the above-mentioned camB originated from Pseudomonas putida. Such a ferredoxin gene also includes functionally equivalent polynucleotide which can be specified in the same manner as in the above-mentioned P-450 gene.

Ferredoxin reductase gene which is incorporated in the expression system of this invention as an essential factor may be originated from bacteria which are xenogenic to host Escherichia coli, and which, under circumstances, may also be xenogenic to the origin of P-450 gene. Concretely, ferredoxin reductase gene originated from any bacteria is usable in this invention so long as the bacteria are capable of co-expression with the above-mentioned P-450 gene and with ferredoxin gene, and so long as the gene encodes ferredoxin reductase which shows the expected activity of P-450 enzyme, i.e., the product of said gene expression of P-450, or, in other words, which catalyzes single oxygen atom insertional reaction against substrate. Examples of such ferredoxin reductase gene, although non-restrictive, include ferredoxin reductase gene originated from Streptomyces coelicolor (hereinafter sometimes referred to as “fdr-1” or “fdr-2”) and putidaredoxin reductase gene originated from the above-mentioned Pseudomonas putida (hereinafter referred to also as camA). Such a gene also includes functionally equivalent polynucleotide which can be specified in the same manner as in the above-mentioned P-450 gene.

In the expression system of this invention, the above-mentioned P-450 gene, ferredoxin gene and ferredoxin reductase gene are introduced in Escherichia coli an operable state. The term “operable state” means that said genes are present in host in such a manner that all of the genes are capable of functional expression. In a typical example of such a state, all of the above-mentioned genes exist in a plasmid, which is capable of autonomous replication in Escherichia coli, together with autonomously replicating sequence, promoter sequence, terminator sequence and drug resistant gene, in a suitable order. Otherwise, all of said genes exist in chromosome of host Escherichia coli such a manner that they are capable of functional expression via a chromosomal DNA integrative vector. The above-mentioned P-450 gene, ferredoxin gene and ferredoxin reductase gene may be arranged in any order in said plasmid. Usually, however, P-450 gene is preferably placed uppermost in the stream. When, in particular, P-450 gene and ferredoxin gene are used as a gene cluster fragment of the same origin, the following orders may be preferable: P-450 gene-ferredoxin gene-ferredoxin reductase gene; P-450 gene-ferredoxin gene-putidaredoxin reductase gene-putidaredoxin gene; or P-450 gene-putidaredoxin reductase gene-putidaredoxin gene.

Plasmid or vector which is usable in the above-mentioned expression system may be capable of stable autonomous replication in Escherichia coli, or may be an integrative vector which is capable of integrating chromosome of Escherichia coli with exogenous gene. Both can be available from those on the market, or by modification where necessary. Plasmids which have a strong promoter for gene transcription are conveniently used for the above-mentioned purpose. Examples of such plasmids include those on the market, such as pET11 and pUC18.

Thus provided actinomycete P-450 gene expression system of this invention is usable for the screening of P-450 enzymes which are suitable for the modification of various kinds of drugs or the bioconversion from precursor into desired drugs, or further for the manufacture of desired drugs from precursor.

### BRIEF EXPLANATION OF DRAWINGS

FIG. 1 shows the structure of plasmid pMoxAB.

FIG. 2 shows the structure of plasmid pMoxAB-fdr1.

FIG. 3 shows the structure of plasmid pMoxAB-fdr2.

FIG. 4 shows the structure of plasmid pMoxAB-camAB.

FIG. 5 shows the structure of plasmid pT7NS-camAB.

FIG. 6 shows the structure of plasmid pCBM-camAB.

FIG. 7 shows the structure of plasmid pSC154A1-camAB.

FIG. 8 shows the structure of plasmid pDoxA1-camAB.

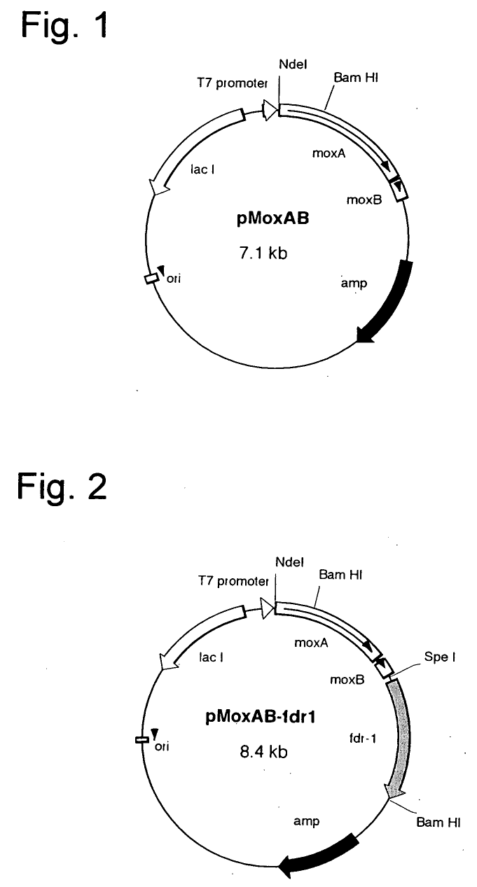

In the following, this invention is concretely explained with working examples.

### BEST MODE FOR WORKING THIS INVENTION

This invention will be further explained with reference to examples of the construction of P-450 gene which forms pravastatin of the following formula: 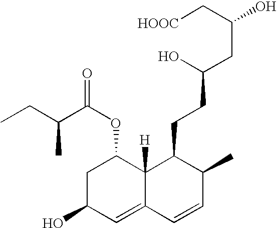
or a mixture (which is called “RT-5.8 substances” in this specification) of isomers thereof which have the following formulae: 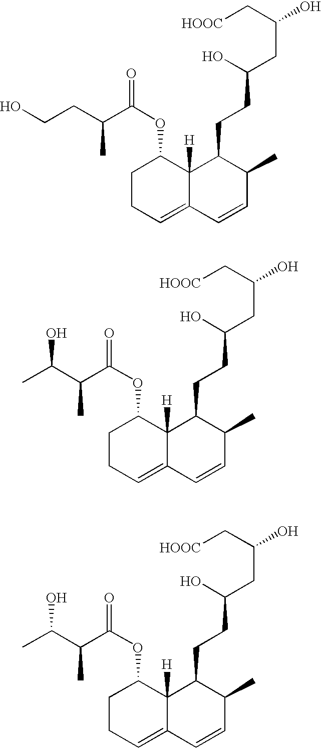
by means of single oxygen atom insertional reaction of compactin of the following formula: 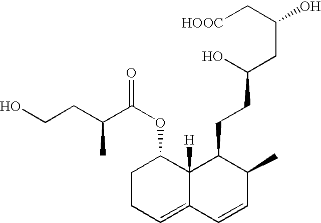
(or also existent as δ-lactone compound corresponding to the above formula) or a salt thereof.

Incidentally, pravastatin sodium is a clinically important medicine as an agent to cure hyperlipidemia.

Actinomycete which has an enzymatic activity to hydroxylate compactin (also called mevastatin) belongs to genus Streptomyces (Japanese Laid-Open (Kokai) Patent Publication Sho 57 (1982)-50894, Japanese Patent No.2672551) or to genus Microtetraspora. As for the latter, which has been confirmed by the inventors of this invention, a DNA fragment which contains P-450 gene can be prepared by the following process.

Preparation of P-450 Gene from Microtetraspora recticatena IFO 14525

Said gene can be obtained by polymerase chain reaction (PCR) with use of primers which have been designed in accordance with an amino acid sequence of the region which is known to keep amino acid sequence with a high probability among a family of lot of P-450 hydroxylation enzymes (J. Bacteriol. 172, 3335-3345 (1990)). For example, IFO 14525 strain is cultivated under certain cultivation conditions, and, then, thus obtained cells are crushed to give chromosomal DNA. Thus obtained chromosomal DNA is subjected to PCR reaction with use of primers which have been designed from amino acid sequences for oxygen-binding domain and heme-binding domain which exist in common with P-450 hydroxylation enzyme family. There is obtained a DNA fragment which has been amplified by the PCR reaction, on the basis of which a further PCR reaction is conducted, and, thus, there is obtained flanking regions of the DNA fragment which has been amplified by the first PCR reaction (in the downstream, there existed a gene which encoded ferredoxin). All of the above-mentioned manipulation can be carried out by any method that is known well in this art. Details of these sets of manipulation are mentioned in the specification of Japanese Patent Application No. 2001-47664 by the same applicant as that of the present application (the contents of said specification are incorporated into the present specification by citation). Sequence No. 1 in the sequence listing shows a nucleotide sequence (and amino acid sequences encoded) which includes adjacent region of thus obtained P-450 gene.

In the above-mentioned sequence, a continuous nucleotide sequence from base 313 to base 1533 corresponds to P-450 gene (moxA), and a continuous nucleotide sequence from base 1547 to base 1741 corresponds to ferredoxin gene (moxB).

Preparation of P-450 Gene from Streptomyces sp. TM-6 or TM-7

From among a lot of microorganisms that belong to Streptomyces which were isolated from the soil in Japan, the applicant has identified the above-captioned TM-6 and TM-7 strains as microorganisms which are capable of biologically converting compactin as a substrate into pravastatin. Said strains were deposited on Apr. 25, 2001, at the International Patent Organism Depository (IPOD) in the National Institute of Advanced Industrial Science and Technology (AIST) at Tsukuba Central 6, 1-1-1 Higashi, Tsukuba, Ibaraki, Japan, and thus have been received with Deposit Nos. FERM P-18311 and FERM P-18312.

Later, a demand was made on the above-mentioned IPOD, which is also an international depositary authority under the Budapest Treaty, for the transfer of these strains TM-6 and TM-7 to said international depositary authority under said Treaty, and, thus, these strains have been received with Deposit Nos. FERM BP-8002 and FERM BP-8003.

With regard to said TM-7 strain, a region of target gene was amplified by PCR in the same manner as in the above-mentioned IFO 14525, and, thus, the sequence of DNA fragment containing the target gene and its adjacent region was determined. The result is shown in Sequence No. 2. In this sequence, a continuous nucleotide sequence from base 544 to base 1758 corresponds to P-450 gene (boxA), and a continuous nucleotide sequence from base 1782 to base 1970 corresponds to ferredoxin gene (boxB). The manipulation to obtain these genes is mentioned detailedly in the specification of Japanese Patent Application No. 2001-166412 which has been filed by the applicant of the present application (the contents of said specification are incorporated into the present specification by citation).

When compared with the sequence of P-450 gene of Streptomyces carbophilus SANK 62585 strain (FERM BP- 1145) which is mentioned, for instance, in Japanese Patent No. 2672551, the nucleotide sequence of the above-mentioned boxA was found to have a homology of about 75%. With the above-mentioned moxA, the boxA has a homology of about 46%. With hydroxylation enzyme gene of Streptomyces lividans, the boxA has a homology of about 75%. Said boxA has a homology of about 46% with a gene encoding pyridylhomothreonine monooxygenase which is an intermediate in the course of biosynthesis of nikkomycin by Streptomyces tendae Tji 901 strain.

On the basis of the above explanation or explanation in working examples mentioned below or, furthermore, on the basis of techniques which are known well in this art or of information in gene database, anyone skilled in the art would be able to obtain various kind of P-450 genes by means of firstly screening actinomycetes which are known (from type culture catalogue published by ATCC) with respect to bio-conversion of substrates for single oxygen atom insertion, then identifying strains having expected enzumatic activity, and thus conducting PCR operation as mentioned above. Hence, actinomycete cytochrome P-450 genes as called in this invention include not only known ones but also all that skilled persons could obtain.

The preparation of ferredoxin gene and ferredoxin reductase gene which are included in the expression system of this invention, and the construction of expression system by means of operable connection between these genes and P-450 genes, could be achieved quite easily by anyone skilled in the art in the same manner as in the above-mentioned P-450 genes, or in accordance with methods as mentioned in literatures (Sambrook et al, Molecular Cloning, A Laboratory Manual, 3^{rd} edition (2001), Cold Spring Harbor Laboratory Press, N.Y.), and in the light of the methods in working examples as mentioned later.

Thus constructed expression system for P-450 genes is capable of functionally expressing P-450 genes under conditions where Escherichia coli is grown. When such an expression system is incubated under a suitable condition together with a substrate for enzyme as a product of P-450 gene (or when transformant as an expression system is cultivated), there is obtained a product wherein single oxygen atom has been inserted in substrate.

Cultivation is usually conducted on a medium which can be a nutritious medium for Escherichia coli, and which has no adverse effects on biological conversion of substrate. Such a medium is composed of suitable carbon source, nitrogen source, inorganic salt and natural organic nutriment. As said carbon source, there can be used glucose, fructose, glycerol, sorbitol and organic acids, either singly or in combination. The concentration of these carbon sources when used is suitably about 1 to 10%, not particularly limited. As said nitrogen source, there can be employed one or two from ammonia, urea, ammonium sulfate, ammonium nitrate and ammonium acetate. As said inorganic salt, there can be used salts such as potassium dihydrogenphosphate, dipotassium hydrogenphosphate, magnesium sulfate, manganese sulfate and ferrous sulfate. As organic nutrient which has growth promoting effects on microorganism used, there can be used peptone, meat extract, yeast extract, corn steep liquor and casamino acids. Furthremore, a small amount of vitamins and nucleic acids may be included in medium.

In the expression system of this invention, high-titer P-450 enzymes with expected activity can be obtained when P-450 enzymes are induced at a temperature of about 25° C. or less, preferably at 20 to 24° C., after host Escherichia coli has been cultivated at a temperature suitable for the growth of Escherichia coli, e.g. at 28 to 40° C.

The following is a detailed explanation of an example of construction of expression system for moxA gene originated from Microtetraspora recticatena IFO 14525 which encodes a compactin-hydroxylating enzyme as an instance of actinomycete cytochrome P-450 enzymes. This invention is, however, not restricted at all by this example.

Polymerase Chain Reaction (PCR):

In the following example, PCT is conducted under conditions as follows.

(1) Condition where genomic DNA is used as a template:

(Composition of reaction liquid)

| | | |
|---|---|---|
| Sterilized purified water | 15 | μl |
| Twice-concentrated GC buffer I (Takara Shuzo) | 25 | μl |
| dNTP mixed solution (dATP, dGTP, dTTP, | 8 | μl |
| dCTP each 2.5 mM) | | |
| Forward primer (100 pmol/μl) | 0.5 | μl |
| Reverse primer (100 pmol/μl) | 0.5 | μl |
| Genomic DNA (10 ng/μl) | 0.5 | μl |
| LA Taq (5 units/μl Takara Shuzo) | 0.5 | μl |

(Temperature condition)
94° C. 3 minutes
(98° C. 20 seconds; 63° C. 30 seconds; 68° C. 2 minutes) 30 cycles
72° C 5 minutes
(2) Condition where plasmid DNA (pMoxAB-fdr1) is used as a template:

(Composition of reaction liquid)

| | | |
|---|---|---|
| Sterilized purified water | 15 | μl |
| Twice-concentrated GC buffer I (Takara Shuzo) | 25 | μl |
| dNTP mixed solution (dATP, dGTP, dTTP, dCTP each 2.5 mM) | 8 | μl |
| Mox-3F primer (100 pmol/μl) | 0.5 | μl |
| Mox-5R primer (100 pmol/μl) | 0.5 | μl |
| Plasmid DNA (1 ng/μl) | 0.5 | μl |
| LA Taq (5 units/μl Takara Shuzo) | 0.5 | μl |

(Temperature condition)
94° C. 3 minutes
(98° C. 20 seconds; 63° C. 30 seconds; 68° C. 2 minutes) 25 cycles
72° C. 5 minutes

### EXAMPLE 1

### Construction of Plasmid

(1) pT7-fdr1

PCR was carried out with use of primer FDR1-1F (5′-GCCATATGACTAGTGCGCCTCACAGACTGGAACGGGAATCTCATG -3′) (see Sequence No.3) and FDR1-2R (5′-GCGAATTCTGTCGGTCAGGCCTGGTCTCCCGTCGGCCG-3′) (see Sequence No. 4) by using, as a template, genomic DNA of Streptomyces coelicolor A3(2) [imparted by John Innes Institute (Norwich, UK)], and, thus, there was amplified a 1.3-kb fragment of gene (hereinafter referred to as fdr-1; see Sequence No. 5) encoding a protein which has homology with ferredoxin reductases. This fragment was treated with restriction enzyme Nde I and Bam HI, and was then subjected to electrophoresis in 0.8% agarose gel. After the electrophoresis was over, the fdr-1 gene fragment was recovered, with use of SUPREC-01 (Takara Shuzo), from a gel piece containing said gene fragment, which had been cut out from the gel, and was purified. Said fragment was ligated to Nde I site and Bam HI site of Escherichia coli plasmid vector pET11a (manufactured by Stratagene Co.) with use of T4 DNA ligase, and, then, Escherichia coli EiDH5α was transformed with the resultant DNA reaction liquid, and, thus, pT7-fdr1 was constructed.

(2) pT7-fdr2

Under the same condition, PCR was carried out with use of primer FDR2-3F (5 ′-CGACTAGTGACGAGGAGGCAGACAAATGGTCGACGCGGATCAG-3 ′) (see Sequence No. 6) and FDR2-4R (5′-CGGGATCCGACAACTATGCGACGAGGCTTTCGAGGG-3′) (see Sequence No. 7) by using genomic DNA of the above-mentioned Streptomyces coelicolor A3(2), and, thus, there was amplified a 1.3-kb fragment of gene (hereinafter referred to as fdr2 see Sequence No. 8), which is different from fdr-1, encoding a protein which has homology with ferredoxin reductases. This fragment was treated with restriction enzyme Bam HI and Spe I, and was then subjected to electrophoresis in 0.8% agarose gel. After the electrophoresis was over, fdr-1 gene fragment was recovered, with use of SUPREC-01 (Takara Shuzo), from a gel piece containing said gene fragment, which had been cut out from the gel, and was purified. Apart from that, plasmid pT7-fdr1 was treated with Bam HI and Spe I, and was then subjected to electrophoresis in 0.8% agarose gel. After the electrophoresis was over, pET11a vector fragment was recovered, with use of SUPREC-01 (Takara Shuzo), from a gel piece containing said vector fragment which had been cut out from the gel, and was purified. Said vector fragment and the above-mentioned fdr-1 gene fragment were ligated to each other with use of T4 DNA ligase, and, then, Escherichia coli DH5α was transformed, and, thus, pT7-fdr2 was constructed.

(3) pT7-camAB

PCR was carried out with use of primer PRR-1F (5′-GCCCCCCATATGAACGCAAACGACAACGTGGTCATC-3′) (see Sequence No. 9) and PRR-2R (5′-GCGGATCCTCAGGCACTACTCAGTTCAGCTTTGGC-3′) (see Sequence No. 10) by using, as a template, genomic DNA of Pseudomonas putida ATCC17453, and, thus, there was amplified a 1.65 kb fragment (camAB fragment; see Sequence No. 16) which contained putidaredoxin reductase gene (camA) and putidaredoxin gene (camB) which was just downstream of said camA. This fragment was treated with restriction enzyme Nde I and Bam HI, and was then subjected to electrophoresis in 0.8% agarose gel. After the electrophoresis was over, camAB fragment was recovered, with use of SUPREC-01 (Takara Shuzo), from a gel piece containing said fragment, which had been cut out from the gel, and was purified. Said fragment was ligated to Nde I site and Bam HI site of Escherichia coli plasmid vector pET11a (manufactured by Stratagene Co.) with use of T4 DNA ligase, and, then, Escherichia coli DH5α was transformed, and, thus, pT7-camAB was constructed.

(4) pMoxAB

PCR was carried out with use of primer Mox-1F (5′-GCCCCCCATATGACGAAGAACGTCGCCGACGAACTG-3′) (see Sequence No. 11) and Mox-12R (5 ′-GCAGATCTAGTGGCTTCAGGCGTCCCGCAGGATGG-3′) (see Sequence No. 12) by using, as a template, genomic DNA of IFO14525 strain, and, thus, there was amplified a 1.4-kb fragment (moxAB fragment) which contained a gene (moxA) encoding compactin-hydroxylation enzyme and ferredoxin gene (moxB) which was adjacent downstream thereto. This fragment was treated with restriction enzyme Nde I and Bgl II I, and was then subjected to electrophoresis in 0.8% agarose gel. After the electrophoresis was over, moxAB fragment was recovered, with use of SUPREC-01 (Takara Shuzo), from a gel piece containing said fragment, which had been cut out from the gel, and was purified. Said fragment was ligated to Nde I site and Bam HI site of the above-mentioned plasmid pET11a with use of T4 DNA ligase, and, then, Escherichia coli DH5α was transformed with resultant reaction liquid, and, thus, plasmid pMoxAB was constructed.

(5) pMoxAB-fdr1 and pMoxAB-fdr2

PCR was carried out with use of primer Mox-1F (5′-GCCCCCCATATGACGAAGAACGTCGCCGACGAACTG-3′) (see Sequence No. 11 as mentioned above) and Mox-2R (5′-CGACTAGTGGCTTCAGGCGTCCCGCAGGATGG-3′) (see Sequence No. 13) by using, as a template, genomic DNA of IFO14525 strain, and, thus, there was amplified a 1.4-kb fragment (moxAB fragment) which contained a gene (moxA) encoding compactin-hydroxylation enzyme and ferredoxin gene (moxB) which was adjacent downstream thereto. This fragment was treated with restriction enzyme Nde I and Spe I, and was then subjected to electrophoresis in 0.8% agarose gel. After the electrophoresis was over, moxAB fragment was recovered, with use of SUPREC-01 (Takara Shuzo), from a gel piece containing said fragment, which had been cut out from the gel, and was purified. Said fragment was ligated to Nde I site and Spe I site of the above-mentioned plasmid pT7-fdr1 with use of T4 DNA ligase, and, then, Escherichia coli DH5α was transformed with resultant reaction liquid, and, thus, plasmid pMoxAB-fdr1 was constructed. FIG. 2 shows the structure of this pMoxAB-fdr1.

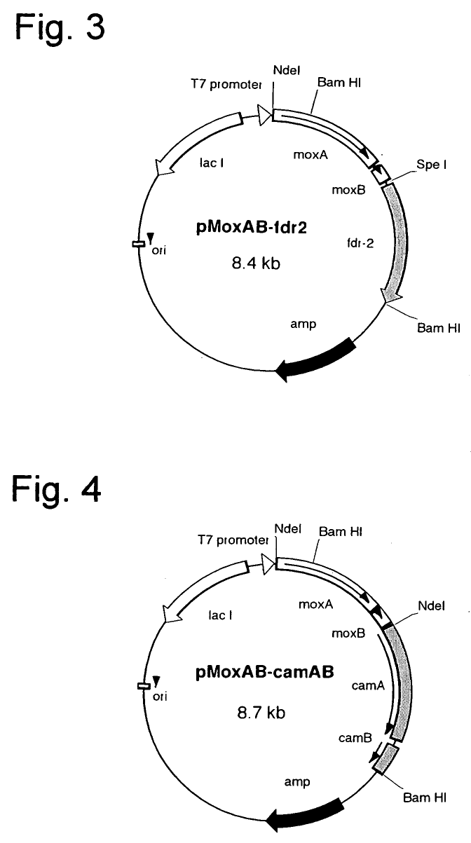

The same inserted fragment was ligated to Nde I site and Spe I site of another plasmid pT7-fdr2 with use of T4 DNA ligase, and, then, Escherichia coli DH5α was transformed with resultant reaction liquid, and, thus, plasmid pMoxAB-fdr2 was constructed. FIG. 3 shows the structure of this pMoxAB-fdr2.

(6) pMoxAB-camAB

PCR was carried out with use of primer Mox-3F (5′-GGAGATATACATATGACGAAGAAC-3′) (see Sequence No. 14) and Mox-5R (5 ′-GCCCCCCATATGACGCACTCCTAGTGGCTTCAGGCGTCCCG-3′) (see Sequence No. 15) by using, as a template, DNA of pMoxAB-fdr1, and, thus, there was amplified a 1.5-kb fragment which contained a gene encoding cytochrome P-450 enzyme having compactin-hydroxylating activity and ferredoxin gene (moxAB) which was adjacent downstream thereto. This fragment was ligated to Nde I site of the plasmid pT7-camAB with use of T4 DNA ligase, and, then, Escherichia coli DH5α was transformed with resultant reaction liquid, and, thus, plasmid pMoxAB-camAB was constructed. FIG. 4 shows the structure of this pMoxAB-camAB.

### EXAMPLE 2

### Preparation of Recombinant Which has Actinomycete Cytochrome P-450 Enzymatic Activity

Escherichia coli BL21(DE3) was transformed with three plasmids, i.e., pMoxAB-fdr1, pMoxAB-fdr2 and pMoxAB-camAB, and, thus, transformant strains corresponding to these plasmids were obtained. Single colony of each of these strains was seeded on 2 ml of LB medium, and was subjected to shake culture at 28° C. for 16 hours at 220 rpm. Thus obtained culture liquid in an amount of 200 μl was mixed with an equal amount (200 μl) of 40% glycerol (sterilized) to give a glycerol culture, which was preserved at −80° C. until used. On the other hand, with use of pMoxAB and pET11a which was used as a vector, Escherichia coli BL21(DE3) was transformed, and, thus, transformant strains corresponding to these plasmids were obtained. Said transformant strains were used as control.

### EXAMPLE 3

### Production of Pravastatin and its Hydroxylated Analogues from Compactin

(1) Production Process with Use of Static Cells:

Glycerol culture of transformant strain of BL21(DE3) as obtained in the above Example 2 in an amount of 10 μd was added to 2 ml of LB medium to which 50 μg/ml (final concentration) of ampicillin had been added, and was then subjected to shake culture at 28° C. for 16 hours at 220 rpm. The resultant culture liquid in an amount of 250 μl was added to 25 ml of NZCYM medium to which 50 μg/ml (final concentration) of ampicillin had been added, and was then subjected to shake culture at 37° C. for 2.5 hours. Then, 25 μl of 100 mM IPTG and 25 μl of 80 mg/ml 5-aminolevulinic acid were added in this order, and the resultant mixture was subjected to shake culture at 18-28° C. (this temperature is hereinafter called as “induction temperature”) for 16 hours at 120 rpm. Cells were recovered by centrifugation from 10 ml of the resultant culture liquid, and were then washed once with conversion buffer-2 (50 mM NaH₂PO₄, 1 mM EDTA, 0.2 mM DTT 10% glycerol, [pH 7.3]). Subsequently, the cells were suspended in 1 ml of said buffer to give a suspension of static cells. To this suspension of static cells, there were added compactin sodium salt (final concentration 250 μg/ml) and NADPH (final concentration 1 mM), and the resultant mixture was incubated at 32° C. for 24-48 hours (this time is hereinafter referred to as “conversion time”) under shaking condition (220 rpm). Later, to 100 μl of thus obtained reaction liquid, there was added 100 μl of acetonitrile, and the resultant mixture was subjected to vortex for one minute at room temperature, and was then centrifuged for 10 minutes at 16,000 rpm with an Epfendorf centrifugator. So obtained supernatant was analyzed with HPLC, and, thus, there were detected pravastatin and other hydroxylated analogues which had been formed by the hydroxylation of substrate compactin. The following shows detailed condition for this HPLC.

Analytical Apparatus: Shimadzu C-4RA Chromatopac

Column: J′ sphere ODS-H80 (YMC, Inc.), 75 mm×4.6 mm I.D.

Mobile phase A: Ion-exchange water/acetic acid/triethylamine=998:1:1 B: Methanol/acetic acid/triethylamine=998:1:1

Gradient time program:

0 minute Mobile phase A/B=50:50

3.00 minute Mobile phase A/B=10:90

3.50 minute Mobile phase A/B=10:90

3.51 minute Mobile phase A/B=50:50

6.00 minute End

| Flow rate: | | 2.0 | ml/minute |
|---|---|---|---|
| Detection: | | UV 237 | nm |
| Injection content: | | 10 | μl |
| Column temperature: | | 40° | C. |
| Analysis time: | | 6 | minutes |
| Retention time: | compactin | 4.2 | minutes |
| | pravastatin | 2.7 | minutes |
| | RT-5.8 substances | 3.6 | minutes |

(2) Production Process by Fed-Batch Method:

Glycerol culture of transformant strain of BL21(DE3) in an amount of 10 μl was added to 2 ml of LB medium to which 50 μg/ml (final concentration) of ampicillin had been added, and was then subjected to shake culture at 28° C. for 16 hours at 220 rpm. The resultant culture liquid in an amount of 250 μl was added to 25 ml of M9-plus medium (M9 salt, 0.4% glucose, 0.5% casamino acids, 100 μg/ml thiamin, 20 μl/ml thymine, 0.1 mM CaCl₂, 1 mM MgCl₂) to which 50 μg/ml (final concentration) of ampicillin had been added, and was then subjected to shake culture at 37° C. for 2.5 hours. Then, 25 μl of 100 mM IPTG and 25 μl of 80 mg/ml δ-aminolevulinic acid were added in this order, and the resultant mixture was subjected to shake culture at 22° C. for 16 hours at 120 rpm. To the resultant culture liquid, there was added 2.5 ml of conversion mixture (2.5 mg/ml compactin sodium salt, 1 mg/ml FeSO₄.7H₂O, 10 mM NADPH, 50% glycerol), and, thus, cultivation was continued at 22° C. for 96 hours. The period of time which has passed after the addition of this conversion mixture is hereinafter referred to as “cultivation time”. Then, to 100 μl of this culture liquid, there was added 100 μl of acetonitrile, and the resultant mixture was subjected to vortex for one minute at room temperature, and was then centrifuged for 10 minutes at 16,000 rpm with an Epfendorf centrifugator. So obtained supernatant was analyzed with HPLC, and, thus, there were detected pravastatin and other hydroxylated analogues which had been formed by the hydroxylation of substrate compactin.

Table 1 shows results of the measurement of the amount of pravastatin and RT-5.8 substance produced by static cells as mentioned in Example 3(1) with use of Escherichia Coli transformant strain, under the protein induction condition of 18-28° C. and with a conversion time of 48 hours.

TABLE 1

| | Induction temperature | | | | | | | |
|---|---|---|---|---|---|---|---|---|
| Hydroxylated | 18° C. | | 22° C. | | 25° C. | | 28° C. | |
| compactin | | RT-5.8 | | RT-5.8 | | RT-5.8 | | RT-5.8 |
| (μg/ml) | Pravastatin | substances | Pravastatin | substances | Pravastatin | substances | Pravastatin | substances |
| BL21(DE3)/ | 0 | 0 | 0 | 0 | 0 | 0 | 0 | 0 |
| pET11a | | | | | | | | |
| BL21(DE3)/ | 0 | 0.18 | 0 | 0 | 0 | 0 | 0 | 0.3 |
| pMoxAB | | | | | | | | |
| BL21(DE3)/ | 0.24 | 1.24 | 1.72 | 8.48 | 0.43 | 1.92 | 0 | 0.61 |
| pMoxAB-fdr1 | | | | | | | | |
| BL21(DE3)/ | 0.25 | 1.31 | 2.15 | 10.21 | 0 | 0.84 | 0.35 | 1.65 |
| pMoxAB-fdr2 | | | | | | | | |
| BL21(DE3)/ | 3.84 | 22.07 | 6.09 | 33.55 | 4.05 | 16.53 | 0.97 | 5.49 |
| pMoxAB-camAB | | | | | | | | |

Productivity was the highest when induction temperature was 22° C., under which condition each of strains wherein Streptomyces coelicolor A3(2)-originated ferredoxin reductase (fdr-1 or fdr-2) had been co-expressed accumulated, in medium, 1.7 to 2.1 μg/ml of pravastatin and 8.4 to 10.2 μg/ml of RT-5.8 substances. A strain wherein camAB had been expressed showed much higher productivity; it accumulated, in medium, 6.09 μg/ml of pravastatin and 33.55 μg/ml of RT-5.8 substance. In the case where there was used, as control, vector alone (BL21(DE3)/pET11a) or a strain which contained no gene to encode ferredoxin reductase (BL21(DE3)/pMoxAB), there were hardly detected pravastatin and RT-5.8 substances.

Table 2 shows results of test of productivity of pravastatin in Fed-batch method as mentioned in Example 4 (2).

TABLE 2

| | Cultivation time | | | | | | | |
|---|---|---|---|---|---|---|---|---|
| Hydroxylated | 4 hours | | 24 hours | | 48 hours | | 96 hours | |
| compactin | | RT-5.8 | | RT-5.8 | | RT-5.8 | | RT-5.8 |
| (μg/ml) | Pravastatin | substances | Pravastatin | substances | Pravastatin | substances | Pravastatin | substances |
| BL21(DE3)/ | 0 | 0 | 0 | 0 | 0 | 0 | 0 | 0 |
| pET11a | | | | | | | | |
| BL21(DE3)/ | 0 | 0 | 0 | 0 | 0 | 0 | 0 | 0 |
| pMoxAB | | | | | | | | |
| BL21(DE3)/ | 0 | 0 | 0 | 0 | 0 | 0.87 | 0 | 0.61 |
| pMoxAB-fdr1 | | | | | | | | |
| BL21(DE3)/ | 0 | 0 | 0 | 0 | 0 | 0.29 | 0 | 0.28 |
| pMoxAB-fdr2 | | | | | | | | |
| BL21(DE3)/ | 0 | 0 | 0.28 | 2.82 | 0.64 | 7.74 | 0.95 | 12.44 |
| pMoxAB-camAB | | | | | | | | |

In the results with cultivation time of 96 hours, strains wherein Streptomyces coelicolor A3(2)-originated ferredoxin reductase (fdr-1 or fdr-2) had been co-expressed accumulated, in medium, 0.28 to 0.61 μg/ml of RT-5.8 substances while accumulating no pravastatin. A strain wherein camAB had been expressed showed high productivity; it accumulated, in medium, 0.95 μg/ml of pravastatin and 12.44 μg/ml of RT-5.8 substances. In the case where there was used, as control, vector alone (BL21(DE3)/pET11a) or a strain which contained no gene to encode ferredoxin reductase (BL21(DE3)/pMoxAB), pravastatin and RT-5.8 substances were not detected.

In a strain wherein camAB had been co-expressed, i.e., in Escherichia coli wherein pMoxAB-camAB had been introduced, moxB (ferredoxin gene) and camB (putidaredoxin gene) among thus introduced genes overlap with each other in their function. In order to know which gene among the constituent genes contained in said pMoxAB-camAB are indispensable for the expression of activity, the inventors of this invention constructed a plasmid which lacked one or two of said constituent genes, and introduced the plasmid into Escherichia Coli. Table 3 shows results of productivity of hydroxylated compactin with use of static cells of thus prepared strain, and with a conversion time of 24 hours.

TABLE 3

| | | | | | Hydroxylated compactin | |
|---|---|---|---|---|---|---|
| | Constituent gene | | | | (μg/ml) | |
| | (+: existent; −: non-existent) | | | | | RT-5.8 |
| | moxA | moxB | camA | camB | Pravastatin | substances |
| BL21(DE3)/ | − | − | − | − | 0 | 0 |
| pET11a | | | | | | |
| BL21(DE3)/ | + | + | − | − | 0 | 0 |
| pMoxAB | | | | | | |
| BL21(DE3)/ | + | + | + | − | 0.17 | 0.82 |
| pMoxAB- | | | | | | |
| camA | | | | | | |
| BL21(DE3)/ | + | − | + | + | 0 | 0 |
| pMoxA- | | | | | | |
| camAB | | | | | | |
| BL21(DE3)/ | + | + | + | + | 1.67 | 9.96 |
| pMoxAB- | | | | | | |
| camAB | | | | | | |

The above shows that three kinds of gene of moxA, moxB and camA are essential for the expression of activity, and that the addition of camB achieves a remarkable increase in activity; the yield of hydroxylated compactin increased about 10 times.

### EXAMPLE 5

### Construction of Plasmid

(1) pT7NS-camAB

PCR was carried out under the following condition with use of primer PRR-1F (5′-GCCCCCCATATGAACGCAAACGACAACGTGGTCATC-3′) (see 15 Sequence No. 9) and PRR-2R (5′-GCGGATCCTCAGGCACTACTCAGTTCAGCTTTGGC-3′) (see Sequence No. 10) by using, as a template, genomic DNA of Pseudomonas putida ATCC17453.

(Composition of reaction liquid)

| | |
|---|---|
| Sterilized purified water | 15 μl |
| Twice-concentrated GC buffer I (Takara Shuzo) | 25 μl |
| dNTP mixed solution (dATP, dGTP, dTTP, dCTP each | 8 μl |
| 2.5 mM) | |
| PRR-1F primer (100 pmol/μl) | 0.5 μl |
| PRR-2R primer (100 pmol/μl) | 0.5 μl |
| Pseudomonas putida ATCC 17453 | |
| Genomic DNA (10 ng/μl) | 0.5 μl |
| LA Taq (5 units/μl Takara Shuzo) | 0.5 μl |

(Temperature condition)
95° C. 3 minutes
(98° C. 20 seconds; 63° C. 30 seconds; 68° C. 2 minutes) 30 cycles
72° C. 5 minutes

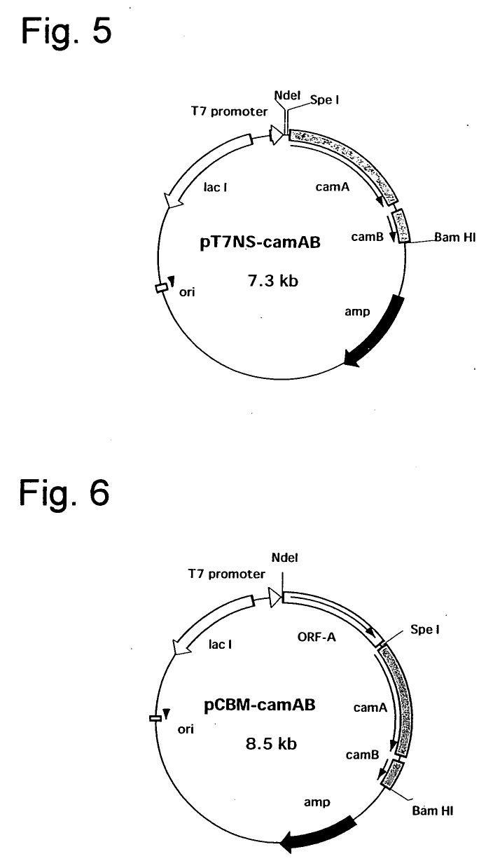

An amplified 1.5-kb fragment (camAB fragment) which contained ferredoxin reductase gene (camA) and putidaredoxin gene (camB just downstream thereto was treated with restriction enzyme Nde I and Bam HI, and was then subjected to electrophoresis in 0.8% agarose gel. After the electrophoresis was over, camAB fragment was recovered, with use of SUPREC-01 (Takara Shuzo), from a gel piece containing said gene fragment which had been cut out from the gel, and was purified. Said fragment was ligated to Nde I site and Bam HI site of Escherichia Coli plasmid vector pET11a (manufactured by Stratagene Co.) with use of T4 DNA ligase, and, then, Escherichia Coli DH5α was transformed, and, thus, pT7-camAB was constructed. To Nde I site of said plasmid pT7-camAB, there was ligated by T4 DNA ligase one molecule of linker which had been prepared by the annealing of two kinds of synthetic oligo DNAs SP-1 (5′-TATGCGTCACTAGTCGGGAGTGCGTTA-3′) (see Sequence No. 17) and SP-2 (5′-TATAACGCACTCCCGACTAGTGACGCA-3′) (see Sequence No. 18), with which Escherichia coli DH5α was transformed, and, thus, plasmid pT7NS-camAB was constructed. FIG. 5 shows the structure of pT7NS-camAB.

(2) pCBM-camAB

PCR was carried out under the following condition with use of primer CB-4F (5′-GCCCCCCATATGACAGCTTTGAATCTGATG-3′) (see Sequence No. 19) and CB-5R (5′-GCACTAGTCAGAGACGGACCGGCAGAC-3′) (see Sequence No. 20) by using, as a template, total DNA of Streptomyces thermotolerans ATCC11416, and, thus, there was prepared 1.25 kb fragment of ORF-A (cytochrome P-450 gene which encodes enzyme to epoxidize 12- and 13-positions of carbomycin B) (gene sequence of ORF-A and the function of ORF-A are mentioned in Bioscience Biotechnology Biochemistry vol.59, 582-588, 1995; the contents of this literature is incorporated into the present specification by this citation).

(Composition of reaction liquid)

| | |
|---|---|
| Sterilized purified water | 61 μl |
| 10 times-concentrated buffer (Takara Shuzo) | 10 μl |
| 25 mMgCl2 | 10 μl |
| dNTP mixed solution (dATP, dGTP, dTTP, dCTP each | 16 μl |
| 2.5 mM) | |
| CB-4F primer (100 pmol/μl) | 0.5 μl |
| CB-5R primer (100 pmol/μl) | 0.5 μl |
| Total DNA (100 ng/μl) of Streptomyces | 1 μl |
| thermotolerans ATCC11416 | |
| LA Taq (5 units/μl, Takara Shuzo) | 1 μl |

(Temperature condition)
95° C. 3 minutes
(98° C. 20 seconds; 63° C. 30 seconds; 68° C. 2 minutes) 30 cycles
72° C. 5 minutes

This gene fragment was digested with restriction enzyme Nde I and Spe I, and was then subjected to electrophoresis in 0.8% agarose gel. After the electrophoresis was over, ORF-A fragment was recovered, with use of SUPREC-01 (Takara Shuzo), from a gel piece containing said gene fragment, which had been cut out from the gel, and was purified. Said fragment was ligated to Nde I-Spe I site of pT7NS-camAB with use of T4 DNA ligase, and, with the resultant reaction liquid, Escherichia Coli DH5α was transformed, and, thus, plasmid pCBM-camAB was constructed. FIG. 6 shows the structure of pCBM-camAB.

(3) pSC154A1-camAB

Total DNA of Streptomyces coelicolor A3(2) (imparted by John Innes Institute, Norwich, UK was digested with restriction enzyme Bam HI and Pst I to give a 100 μg/μl solution of TE (10 mM Tris-HCl [pH 8.0], 1 mM EDTA). PCR was carried out under the following condition by using this DNA as a template with use of primer 154A1-1F (5′-GCCCCCCATATGGCGACCCAGCAGCCCGCCCTC-3′) (see Sequence No. 21) and 154A1-2R (5′-GCACTAGTCAGCCGGCGTGCAGCAGGACCGG-3′) (see Sequence No. 22), and, thus, there was prepared 1.2 kb gene fragment which encoded CYP154A1 (DNA sequence of gene which encodes Streptomyces coelicolor A3(2)-originated CYP154A1 has been published in gene database, e.g., Gen Bank, under gene name of SCE6.21).

(Composition of reaction liquid)

| | |
|---|---|
| Sterilized purified water | 61 μl |
| 10 times-concentrated buffer (Takara Shuzo) | 10 μl |
| 25 mMgCl2 | 10 μl |
| dNTP mixed solution (dATP, dGTP, dTTP, dCTP each | 16 μl |
| 2.5 mM) | |
| 154A1-1F primer (100 pmol/μl) | 0.5 μl |
| 154A1-2R primer (100 pmol/μl) | 0.5 μl |
| Total DNA (100 ng/μl) of Streptomyces | 1 μl |
| coelicolor A3(2) digested with | |
| Bam HI-PstI | |
| LA Taq (5 units/μl, Takara Shuzo) | 1 μl |

(Temperature condition)
95° C. 3 minutes
(98° C. 20 seconds; 63° C. 30 seconds; 68° C. 2 minutes) 30 cycles
72° C. 5 minutes

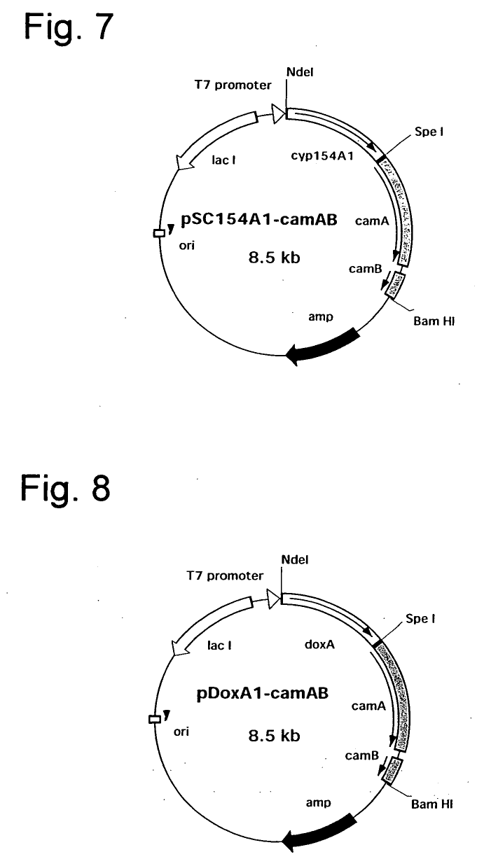

This gene fragment was digested with restriction enzyme Nde I and Spe I, and was then subjected to electrophoresis in 0.8% agarose gel. After the electrophoresis was over, CYP154A1-encoding gene fragment was recovered, with use of SUPREC-01 (Takara Shuzo), from a gel piece containing said gene fragment which had been cut out from the gel, and was purified. Said fragment was ligated to NdeI-SpeI site of pT7NS-camAB with use of T4 DNA ligase, and, with the resultant reaction liquid, Escherichia Coli DH5α was transformed, and, thus, plasmid pSC154A1-camAB was constructed. FIG. 7 shows the structure of pSC154A1-camAB. ps (4) pDoxA1-camAB

PCR was carried out under the following condition with use of primer DoxA-1F (5′-GCCCCCCATATGGCCGTCGACCCGTTCGCGTG-3′) (see Sequence No. 23) and DoxA-2R (5′-GCACTAGTCAGCGCAGCCAGACGGGCAGTTC-3′) (see Sequence No. 24) by using, as a template, total DNA of daunomycin-producing bacterium Streptomyces peucetius ATCC 29050, and, thus, there was prepared 1.2-kb fragment of doxA (cytochrome P-450 gene which participates in the biosynthesis of daunomycin). DNA sequence of the doxA gene is mentioned in Journal of Bacteriology, vol. 181, No. 1, 305-318, 1999 (the contents of this literature is incorporated into the present specification by this citation).

(Composition of reaction mixture)

| | |
|---|---|
| Sterilized purified water | 61 μl |
| 10 times-concentrated buffer (Takara Shuzo) | 10 μl |
| 25 mMgCl2 | 10 μl |
| dNTP mixed solution (dATP, dGTP, dTTP, dCTP each 2.5 mM) | 16 μl |
| DoxA-1F primer (100 pmol/μl) | 0.5 μl |
| DoxA-2R primer (100 pmol/μl) | 0.5 μl |
| Total DNA (100 ng/μl) of Streptomyces peuceticus ATCC 29050 | 1 μl |
| LA Taq (5 units/μl, Takara Shuzo) | 1 μl |

(Temperature condition)
95° C. 3 minutes
(98° C. 20 seconds; 63° C. 30 seconds; 68° C. 2 minutes) 30 cycles
72° C. 5 minutes

This gene fragment was digested with restriction enzyme Nde I and Spe I, and was then subjected to electrophoresis in 0.8% agarose gel. After the electrophoresis was over, doxa fragment was recovered, with use of SUPREC-01 (Takara Shuzo), from a gel piece containing said gene fragment which had been cut out from the gel, and was purified. Said fragment was ligated to Nde I-Spe I site of pT7NS-camAB with use of T4 DNA ligase, and, with the resultant reaction liquid, Escherichia Coli DH5α was transformed, and, thus, plasmid pDoxA1-camAB was constructed. FIG. 8 shows the structure of pDoxA1-camAB.

### EXAMPLE 6

### Microbial Conversion with Use of Escherichia coli Recombinant Wherein Cytochrome P-450 Had Been Expressed

(1) Production of carbomycin A

Glycerol culture, in an amount of 10 μl, of Escherichia coli BL21(DE3) strain which had been transformed with pCBM-camAB among the plasmids as obtained in the above-mentioned Example 4 was added to 2 ml of LB medium to which 50 μg/ml (final concentration) of ampicillin had been added, and the resultant mixture was subjected to shake culture at 28° C. for 16 hours at 220 rpm. Thus obtained culture liquid in an amount of 250 μl was added to 25 ml of NZCYM medium to which 50 μg/ml of ampicillin had been added, and the resultant mixture was subjected to shake culture at 37° C. for 2.5 hours. Then, 25 μl of 100 mM IPTG and 25 μl of 80 mg/ml δ-aminolevulic acid were added in order, and the resultant mixture was subjected to shake culture at 22° C. at 120 rpm for 16 hours. Cells were recovered by centrifugation from the resultant culture liquid, and were then washed once with conversion buffer-2 (50 mM NaH₂PO₄, 1 mM EDTA, 0.2 mM DTT, 10% glycerol, [pH 7.3]). Subsequently, the cells were suspended in 3 ml of said buffer to give a suspension of static cells. To 600 μl of this suspension of static cells, 3 μl of 100 mg/ml methanol solution of carbomycin B was added, and the resultant mixture was incubated at 28° C. for five hours with shaking (220 rpm). Later, to thus obtained reaction liquid, there was added 100 μl of 50% K2HPO₄ (pH 8.5) and 100 μl of ethyl acetate, and the resultant mixture was subjected to vortex, and then to centrifugation for five minutes at 16,000 rpm with an Epfendorf centrifugator. A TLC plate was spotted with 10 μl of so obtained ethyl acetate phase, and was then subjected to development with a developer (ethyl acetate: diethylamine=100:2). Subsequently this plate was sprayed with 10% sulfuric acid, and heated at 100° C. for 10 minutes. Spots on which color had come out were analyzed for coloring intensity with a dual-wavelength chromatoscanner CS-930 (manufactured by Shimadzu Seisakusho) at a wavelength of 600 nm, and, thus, there was evaluated the amount of carbomycin A (RF value in TLC: 0.64) which had been formed by the epoxidation of substrate carbomycin B (RF value in TLC: 0.71). As a result, it was confirmed that carbomycin A had been formed with a yield of 90 μg/ml. Then, substrate conversion reaction was conducted with use of a control strain BL21(DE3) (pET11a) under the same condition as stated above. As a result of analysis, no formation of carbomycin A was detected.

(2) De-ethylation of 7-ethoxycoumarin

Glycerol culture, in an amount of 10 μl, of Escherichia Coli BL21(DE3) strain which had been transformed with pSC154A1-camAB among the plasmids as obtained in the above-mentioned Example 5 was added to 2 ml of LB medium to which 50 μg/ml (final concentration) of ampicillin had been added, and the resultant mixture was subjected to shake culture at 28° C. for 16 hours at 220 rpm. Thus obtained culture liquid in an amount of 250 μl was added to 25 ml of NZCYM medium to which 50 μg/ml of ampicillin had been added, and the resultant mixture was subjected to shake culture at 37° C. for 2.5 hours. Then, 25 μl of 100 mM IPTG and 25 μl of 80 mg/ml δ-aminolevulinic acid were added in order, and the resultant mixture was subjected to shake culture at 22° C. at 120 rpm for 16 hours. Cells were recovered by centrifugation from the resultant culture liquid, and were then washed once with conversion buffer-2 (50 mM NaH₂PO₄, 1 mM EDTA, 0.2 mM DTT, 10% glycerol, [pH 7.3]). Subsequently, the cells were suspended in 6 ml of said buffer to give a suspension of static cells. To 1 ml of this suspension of static cells, 5 μl of 50 mM DMSO solution of 7-ethoxycoumarin was added, and the resultant mixture was incubated at 28° C. for five hours with shaking (220 rpm). Later, to thus obtained reaction liquid, there was added 200 μl of ethyl acetate, and the resultant mixture was subjected to vortex, and then to centrifugation for five minutes at 16,000 rpm with an Epfendorf centrifugator. There was taken out 100 μl of so obtained ethyl acetate phase, which was then evaporated to dryness in vacuum. The resultant dried pellet was dissolved in 1 ml of 100 mM potassium phosphate buffer (pH 7.4). Thus obtained solution was 80-times diluted, and was then measured for fluorescence (wavelength: 460 nm) with F-2000 spectrophotofluorometer (manufactured by Hitachi Science Systems, Co.) at an excitation wave length of 380 nm for the purpose of evaluation of the amount of 7-hydroxycoumarin which had been formed by de-ethylation of substrate 7-ethoxycoumarin. As a result, fluorescence intensity was 2770, and, thus, the formation of 7-hydroxycoumarin was confirmed. Then, substrate conversion reaction was conducted with use of a control strain BL21(DE3) (pET11a) under the same condition as stated above. As a result of analysis, fluorescence intensity was three or less.

(3) Dehydrogenation of 13-dihydrodaunomycin

Glycerol culture, in an amount of 10 μl of Escherichia coli BL21(DE3) strain which had been transformed by pDoxA1-camAB among the plasmids as obtained in the above-mentioned Example 5 was added to 2 ml of LB medium to which 50 μg/ml (final concentration) of ampicillin had been added, and the resultant mixture was subjected to shake culture at 28° C. for 16 hours at 220 rpm. Thus obtained culture liquid in an amount of 250 μl was added to 25 ml of NZCYM medium to which 50 μg/ml of ampicillin had been added, and the resultant mixture was subjected to shake culture at 37° C. for 2.5 hours. Then, 25 μl of 100 mM IPTG and 25 μl of 80 mg/ml δ-aminolevulinic acid were added in order, and the resultant mixture was subjected to shake culture at 22° C. at 120 rpm for 24 hours. Cells were recovered by centrifugation from the resultant culture liquid, and were then washed once with conversion buffer-2 (50 mM NaH₂PO₄, 1 mM EDTA, 0.2 mM DTT, 10% glycerol, [pH 7.3]). Subsequently, the cells were suspended in 4 ml of said buffer to give a suspension of static cells. To 1 ml of this suspension of static cells, 10 μl of 10 mg/ml methanol solution of 13-dihydrodaunomycin was added, and the resultant mixture was incubated at 28° C. for 24 hours with shaking (220 rpm). Later, to 400 μl of thus obtained reaction liquid, there was added 1.2 ml of acetone, and the resultant mixture was subjected to vortex, and was then extracted with 300 μl of chloroform. Thus obtained extract was evaporated to dryness in vacuum, and was then dissolved in 500 μl of 0.3 M hydrochloric acid, and thus obtained solution was heated at 80° C. for 30 minutes. This solution was extracted with 100 μl of chloroform, and thus obtained extract was evaporated to dryness in vacuum. The resultant dried pellet was dissolved in 100 μl of methanol, and the resultant solution was subjected to HPLC under the following condition, and, thus, there was detected daunomycin which had been formed by the dehydrogenation of substrate 13-dihydrodaunomycin.

(Analytical condition of HPLC)

Analytical apparatus: Shimadzu LC10 Chromatopac (manufactured by Shimadzu Seisakusho)

Column: ZORBAX TMS (5 μl) 4.6 mm×250 mm I.D.

Mobile phase: To a mixture of water/acetonitrile/methanol/phosphoric acid=540:290:170:2 (volume ratio), 1.0 g of sodium lauryl sulfate was added and dissolved, and, to the resultant mixture, 2N NaOH was added for the adjustment of pH to 3.6.

| Flow rate: | | 1.5 | ml/minute |
|---|---|---|---|
| Wavelength for detection: | | 254 | nm |
| Injection content: | | 20 | μl |
| Column temperature: | | 40° | C. |
| Analysis time: | | 20 | minutes |
| Retention time: | 13-dihydrodaunomycin | 4.8 | minutes |
| | daunomycin | 5.9 | minutes |

Analysis detected 3.7 μg/ml of daunomycin. Then, substrate conversion reaction was conducted with use of a control strain BL21(DE3) (pET11a) under the same condition as stated above. As a result of analysis, no formation of daunonycin was detected.

### INDUSTRIAL APPLICABILITY

In accordance with this invention, single oxygen atom insertional reaction of organic compound as a substrate can efficiently be conducted with use of a recombinant which has been constructed by use of actinomycete cytochrome P-450 gene and Escherichia Coli a host.

This invention also provides a gene library suitable as an objective of high throughput screening or other simple and rapid enzymatic assay screening for the screening of industrially important and desired actinomycete P-450 enzymes.

1. A system for the expression of actinomycete cytochrome P-450 genes in host Escherichia coli, wherein said Escherichia Coli supports a recombinant DNA molecule which comprises xenogenic microorganism-originated ferredoxin gene, ferredoxin reductase gene as well as said cytochrome P-450 gene, in operable state.

2. An expression system of claim 1 wherein ferredoxin gene and ferredoxin reductase gene are independently originated from some strain of actinomycete.

3. An expression system of claim 1 wherein ferredoxin gene is originated from microorganism selected from the group consisting of those belonging to genus Microtetraspora and those belonging to genus Pseudomonas.

4. An expression system of claim 1 wherein ferredoxin reductase gene is originated from microorganism selected from the group consisting of those belonging to genus Streptomyces and those belonging to genus Pseudomonas.

5. An expression system of claim 1 wherein actinomycete cytochrome P-450 gene and ferredoxin gene are originated from one and the same gene cluster of actinomycete.

6. An expression system of claim 1 wherein ferredoxin reductase gene is originated from Streptomyces coelicolor.

7. An expression system of claim 1 wherein actinomycete cytochrome P-450 gene and ferredoxin gene are originated from one and the same gene cluster of actinomycete, and wherein ferredoxin reductase gene is originated from Streptomyces coelicolor.

8. An expression system of claim 1 wherein ferredoxin gene and ferredoxin reductase gene are respectively putidaredoxin gene (camB and putidaredoxin reductase gene (camA) which are each originated from Pseudomonas putida.

9. An expression system of claim 1 in which actinomycete cytochrome P-450 gene and ferredoxin gene are originated from one and the same gene cluster of actinomycete, and which further contains, as another ferredoxin gene, putidaredoxin gene (camB originated from Pseudomonas putida.

10. An expression system of claim 1 in which actinomycete cytochrome P-450 gene and ferredoxin gene are originated from one and the same gene cluster of actinomycete, in which ferredoxin reductase gene is putidaredoxin reductase gene (camA) originated from Pseudomonas putida, and which further contains, as another ferredoxin gene, putidaredoxin gene (camB) originated from Pseudomonas putida.

11. An expression system of claim 1 wherein actinomycete cytochrome P-450 gene and ferredoxin gene are respectively compactin-hydroxylating enzyme-encoding gene (moxA) originated from Microtetraspora recticatina and ferredoxin gene (moxB) which is adjacent downstream to moxA.

12. An expression system of claim 1 wherein actinomycete cytochrome P-450 gene and ferredoxin gene are respectively compactin-hydroxylating enzyme-encoding gene (moxA) originated from Microtetraspora recticatina and ferredoxin gene (moxB) which is adjacent downstream to moxA, and wherein ferredoxin reductase gene is ferredoxin reductase gene fdr-1 or fdr-2 originated from Streptomyces coelicolor.

13. An expression system of claim 1 in which actinomycete cytochrome P-450 gene and ferredoxin gene are respectively compactin-hydroxylating enzyme-encoding gene (moxA originated from Microtetraspora recticatina and ferredoxin gene (moxB) adjacent downstream to moxA, and which further contains, as another ferredoxin gene, putidaredoxin gene (camB) originated from Pseudomonas putida, and in which ferredoxin reductase gene is putidaredoxin reductase gene (camA) originated from Pseudomonas putida.

14. An expression system of claim 1 in which the induction of expression of cytochrome P-450 gene is conveniently carried out at 20 to 24° C.

15. An expression system of claim 1 wherein said cytochrome P-450 gene comprises polynucleotide which is selected from the group consisting of polynucleotide having a continuous nucleotide sequence from base 313 to base 1533 in Sequence No. 1 or functionally equivalent polynucleotide with homology of at least 80% to said nucleotide sequence, and polynucleotide having a continuous nucleotide sequence from base 544 to base 1758 in Sequence No. 2 or functionally equivalent polynucleotide with homology of at least 80% to said nucleotide sequence.

16. An expression system of claim 1 wherein said ferredoxin gene comprises polynucleotide which is selected from the group consisting of polynucleotide having a continuous nucleotide sequence from base 1547 to base 1741 in Sequence No. 1 or functionally equivalent polynucleotide with homology of at least 80% to said nucleotide sequence, and polynucleotide having a continuous nucleotide sequence from base 1782 to base 1970 in Sequence No. 2 or functionally equivalent polynucleotide with homology of at least 80% to said nucleotide sequence.

17. An expression system of claim 1 wherein said ferredoxin reductase gene comprises polynucleotide which is selected from the group consisting of polynucleotide having a continuous nucleotide sequence from base 118 to base 1377 in Sequence No. 5 or functionally equivalent polynucleotide with homology of at least 80% to said nucleotide sequence, and polynucleotide having a continuous nucleotide sequence from base 34 to base 1296 in Sequence No. 8 or functionally equivalent polynucleotide with homology of at least 80% to said nucleotide sequence.

18. An expression system of claim 1 wherein said ferredoxin gene comprises polynucleotide having a continuous nucleotide sequence from base 1439 to base 1759 in Sequence No. 16 or functionally equivalent polynucleotide with homology of at least 80% to said nucleotide sequence.

19. An expression system of claim 1 wherein said ferredoxin reductase gene comprises polynucleotide having a continuous nucleotide sequence from base 115 to base 1380 in Sequence No. 16 or functionally equivalent polynucleotide with homology of at least 80% to said nucleotide sequence.

20. A method to introduce a hydroxyl group at 6β- position of compactin with use of the expression system of claim 12 or 13.
